(19) United States
(12) Patent Application Publication
(10) Pub. No.: US 2016/0144128 A1
(43) Pub. Date: May 26, 2016

(54) COUNTING MECHANISM FOR A DRUG DELIVERY DEVICE

(71) Applicant: SANOFI, Paris (FR)

(72) Inventors: Tom Oakley, Cambridge Cambridgeshire (GB); Matt Schumann, Bourn Cambridge, Cambridgeshire (GB)

(21) Appl. No.: 14/905,129

(22) PCT Filed: Jul. 17, 2014
(86) PCT No.: PCT/EP2014/065326

§ 371 Date: Jan. 14, 2016

(30) Foreign Application Priority Data
Jul. 17, 2013 (EP) 13176843.4

(51) Int. Cl. A61M 5/315

(52) U.S. Cl. CPC A61M 5/31568; A61M 5/3155

(57) ABSTRACT

The present disclosure relates to a counting mechanism (2) for a drug delivery device (1) comprising a first counting member (4) arranged for displaying a counted number and being biased to move in a first rotational direction and a connecting member (5) having a dose setting position and a dose dispensing position, wherein, in the dose setting position, the connecting member (5) impedes a rotation of the first counting member (4) in the first rotational direction, and wherein, in the dose dispensing position, the connecting member (5) permits the first counting member (4) to rotate in the first rotational direction.

This disclosure relates to a counting mechanism for a drug delivery device and to a drug delivery device comprising such a counting mechanism.

It is an object of the present disclosure to provide an improved counting mechanism for a drug delivery device, e.g. a counting mechanism having a low number of parts. Moreover, it is an object of the present disclosure to provide an improved drug delivery device.

This object may, inter alia, be achieved by the subject of claim 1. Advantageous embodiments and refinements are the subject-matter of the dependent claims. However, further advantageous concepts may be disclosed herein besides the ones which are claimed.

One aspect relates to a counting mechanism for a drug delivery device comprising a first counting member arranged for displaying a counted number and being biased to move in a first rotational direction and a connecting member having a dose setting position and a dose dispensing position, wherein, in the dose setting position, the connecting member impedes a rotation of the first counting member in the first rotational direction, and

wherein, in the dose dispensing position, the connecting member permits the first counting member to rotate in the first rotational direction.

The drug delivery device may be an injection device. The device may be a disposable or a re-usable device. The device may be configured to dispense variable, preferably user-settable, doses of the drug. Alternatively, the device may be a fixed dose device, in particular a device configured to dispense doses of the drug which may not be varied by the user. The drug delivery device may be a manually, in particular a non-electrically, driven device.

The counting mechanism is a mechanism that is configured to provide information to the user. In particular, the first counting member may provide the information to the user by displaying the counted number. The counted number may correspond to the number of currently set doses. Accordingly, the counted number may be incremented or decremented during a dose setting operation of the drug delivery device. The counted number may be decremented during a dose dispensing operation of the drug delivery device. Alternatively, the counted number may correspond to the number of doses remaining in the drug delivery device or to the number of doses that have already been dispensed by the drug delivery device.

The first counting member may comprise a first counting wheel having digits on one of its surfaces displaying the counted number. In particular, the counting mechanism may further comprise a second counting wheel also having digits on one of its surfaces wherein one digit of each counting wheel may be visible in a display. Thus, the counting wheels may cooperate to display the counted number. The counting wheels may be coupled with each other by a gearing member transferring a rotation of the first counting wheel into a movement of the second counting wheel.

The first counting member is biased to move in the first rotational direction. The first rotational direction may be a rotational direction of a rotation around a longitudinal axis of the drug delivery device. As the first counting member is biased to move in the first rotational direction, it may be configured to rotate in the first rotational direction if no other forces are applied to the first counting member by the counting mechanism or by other elements of the drug delivery device. In particular, the first counting member may rotate in the first rotational direction due to the biasing when the connecting member does not impede this rotation.

The connecting member may be configured to connect the first counting member with a drive mechanism. In particular, the connecting member may connect the first counting member with a drive member of the drug delivery device in the dose dispensing position. Thus, due to the biasing, the first counting member may rotate in the first rotational direction when permitted by the connecting member. This rotation may be transferred to the drive member by the connecting member such that the drive member may also rotate. Thus, the counting mechanism may be configured to cooperate with the drive mechanism. Accordingly, elements of the counting mechanism, e.g. the first counting member and/or the connecting member, may also serve a function in the drive mechanism. Thereby, the total number of parts of the drug delivery device may be reduced.

The connecting member may be configured to be in its dose setting position during a dose setting operation of the drug delivery device. Further, the connecting member may be configured to be in its dose setting position in a rest state of the drug delivery device wherein no dose is set. The connecting member may be configured to be in its dose dispensing position during a dose dispensing operation of the drug delivery device. Moreover, the connecting member may be configured such that it returns from the dose dispensing position to the dose setting position at the end of the dose dispensing operation when the set dose is completely dispensed. Further, the connecting member may be configured such that it moves from the dose setting position to the dose dispensing position when the user initiates a dose dispensing operation, e.g. by pressing a trigger member.

The connecting member impeding a rotation of the first counting member in the first rotational direction in the dose setting position may correspond to the connecting member preventing that the first counting member rotates in the first rotational direction due to the biasing of the first counting member alone. Nevertheless, the first counting member may be rotated in the first rotational direction by other means, e.g. if the user applies a force. The user may apply a force e.g. by rotating a control member being connected to the connecting member, thereby temporarily releasing the impediment of the connecting member against a rotation of the first counting member. This may occur during a dose setting operation when the user reduces the set dose.

The connecting member permitting the first counting member to rotate in the first rotational direction in the dose dispensing position may correspond to the connecting member allowing that the first counting member is rotated in the first rotational direction due to the biasing alone.

In one embodiment, the connecting member may permit a rotation of the first counting member in a second rotational direction in the dose setting position. A rotation of the first counting member in the second rotational direction may correspond to incrementing of the counted number. Accordingly, a rotation of the first counting member in the first rotational direction may correspond to decrementing the counted number.

The connecting member may be rotationally locked with the first counting member in the dose setting position and in the dose dispensing position. Accordingly, if the user rotates the connecting member during a dose setting operation, the first counting member is also rotated. Further, if the first counting member is rotated in the first rotational direction in the dose dispensing position of the connecting member due to the biasing of the first counting member, the connecting member is also rotated together with the first counting member.

In one embodiment, the connecting member may be movable along a longitudinal axis of the mechanism between the dose setting position and the dose dispensing position. The dose setting position may be a more proximal position than the dose dispensing position.

The drug delivery device has a distal end and a proximal end. The term “distal end” designates that end of the drug delivery device or a component thereof which is or is to be arranged closest to a dispensing end of the drug delivery device. The term “proximal end” designates that end of the device or a component thereof which is or is to be arranged furthest away from the dispensing end of the device. Accordingly, the “distal direction” is the direction from proximal end towards distal end and the “proximal direction” is direction from distal end towards proximal end.

Further, the counting mechanism may comprise a trigger member configured to move the connecting member from the dose setting position to the dose dispensing position when the trigger member is operated. The trigger member may comprise a button. The trigger member may be operated by pressing the button. The trigger member may abut the connecting member in the dose setting position. When the trigger member is operated, it may move in the distal direction. Due to the abutment with the connecting member, the trigger member may move the connecting member into the dose dispensing position when the trigger member is pushed distally. Thus, the trigger member may be configured to initiate the dose dispensing operation when operated.

Further, the counting mechanism may comprise a return member tending to move the connecting member to the dose setting position. Thus, the connecting member may be moved to the dose setting position at the end of the dose dispensing operation by the return member.

The return member may comprise a spring. The spring may be arranged between a body of the drug delivery device and the connecting member. The spring may be compressed when the connecting member is in the dose dispensing position.

Further, the counting mechanism may comprise a spring member which biases the first counting member to move in the first rotational direction. The spring member may comprise a torsion spring which is tensed when the first counting member is rotated in the second rotational direction. Accordingly, the torsion spring may be configured to release its tension when the first counting member is rotated in the first rotational direction.

Thus, energy may be stored when the first counting member is rotated in the second rotational direction. Further, the mechanism may comprise an energy storing member which is configured to store energy when the first counting member is rotated in the second rotational direction. Further, the energy storing member may be configured to release the stored energy and thereby to move the first counting member in the first rotational direction when the connecting member is in the dose dispensing position.

The spring member may be identical with said energy storing member. However, in an alternate design, the energy storing member may be an element separate from the spring member. The energy storing member may be e.g. a second spring member coupled to the first counting member or a completely different element configured to store energy.

Thus, the spring member may serve two functions. The spring member may be configured to reset the first counting member to a “zero” position during the dose dispensing operation as the spring member rotates the first counting member in the first rotational direction during the dose dispensing operation. Further, as the spring member stores energy when the first counting member is rotated in the second rotational direction during the dose setting operation, the spring member may be configured to use the stored energy to drive a drive member of the drug delivery device via the first counting member during the dose dispensing operation. The spring member may be configured to drive the drive member such that moves a piston rod in the distal direction.

Further, the counting mechanism may comprise a last dose member being engaged with the connecting member in the dose setting position and in the dose dispensing position, wherein the last dose member is configured to move relative to the connecting member when the connecting member is rotated in the dose setting position. The connecting member may comprise a last dose member stop, and a rotation of the connecting member in the second rotational direction is prevented when the last dose member abuts the last dose member stop. Thus, the last dose member and the last dose member stop may be configured to prevent that a dose is set which is greater than the amount of the drug remaining in the drug delivery device.

According to one aspect, the present disclosure refers to a drug delivery device comprising a counting mechanism as disclosed above and a body. In particular, said counting mechanism may be arranged inside the body of the drug delivery device. The body may be an inner body or an outer housing.

In particular, the drug delivery device may comprise the counting mechanism as disclosed above. Accordingly, every structural and functional feature disclosed with respect to that counting mechanism may also be present in the drug delivery device.

In one embodiment, the drug delivery device may comprise a piston rod and a drive member configured to move the piston rod, wherein the connecting member is engaged with the drive member in the dose dispensing position such that the drive member is rotationally locked with the connecting member in the dose dispensing position.

The drive member may comprise a drive nut having a thread at its inner surface. The piston rod may comprise a lead screw having a thread at its outer surface. The drive member and the piston rod may be threadedly engaged. The drive member and the piston rod may be engaged such that a rotation of the drive member relative to the piston rod is transferred into a translational movement of the piston rod.

The connecting member may be configured to engage with the drive member such that the connecting member and the drive member are prevented from rotating relative to each other and are permitted to move translational relative to each other. Thus, when the connecting member is in the dose dispensing position, the drive member may follow a rotation of the first counting member as the connecting member may be rotationally locked to the first counting member and to the drive member in the dose dispensing position.

The connecting member may be disengaged from the drive member in the dose setting position such that the connecting member is permitted to rotate relative to the drive member in the dose setting position. Thus, when the connecting member is in the dose setting position, the drive member may not follow a rotation of the first counting member as the connecting member may not be rotationally locked to the drive member in the dose setting position.

The connecting member may be engaged with the body in the dose setting position. In particular, the body may comprise an engagement member. The engagement member may be e.g. a detent. The engagement of the connecting member and the body may be configured such that it allows the connecting member to be rotated freely in the second rotational direction and that it impedes a rotation of the connecting member in the first rotational direction. Thereby, it may also impede a rotation of the first counting member in the first rotational direction as the first counting member may be rotationally locked with the connecting member.

The connecting member may be disengaged from the body in the dose dispensing position. Thus, in the dose dispensing position, the connecting member and the first counting member may be rotated freely in the first rotational direction.

The drug delivery device may comprise a distal end portion, a gripping portion and a proximal end portion wherein the gripping portion is a portion of a maximum diameter in the drug delivery device, and wherein the gripping portion is arranged between the distal end portion and the proximal end portion.

The gripping portion having a larger diameter than the other portions provides an ergonomic grip for operating the drug delivery device. The gripping portion may also allow for various alternative gripping positions of the device which may be helpful when operating the drug delivery device to carry out an injection at a site which is difficult to access.

In particular, the maximum diameter of the gripping portion may be at least 10% bigger than the maximum diameter of the distal end portion and the maximum diameter of the proximal end portion. Preferably, the maximum diameter of the gripping portion may be at least 20% bigger than the maximum diameter of the distal end portion and the maximum diameter of the proximal end portion. Thereby it may be ensured that the gripping portion provides an ergonomic grip for operating the drug delivery device.

Further, at least one of the first counting member, the spring member and the drive member may be arranged completely inside the gripping portion. Thereby, the total length of the drug delivery device may be significantly reduced. The reduction of the length may be at the expense of an increased diameter. However, the drug delivery device with a reduced hand is easier to operate for users with small hands. Thus, reducing the length of the drug delivery device makes the drug delivery device usable for more users.

The term “drug”, as used herein, preferably means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly- Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropin (Follitropin, Lutropin, Choriongonadotropin, Menotropin), Somatropin (Somatropin), Desmopressin, Terlipressin, Gonadorelin, Triptorelin, Leuprorelin, Buserelin, Nafarelin, Goserelin.

A polysaccharide is for example a glucosaminoglycan, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCI or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

In the following, the disclosed devices and methods are described in further detail with reference to the drawings, wherein

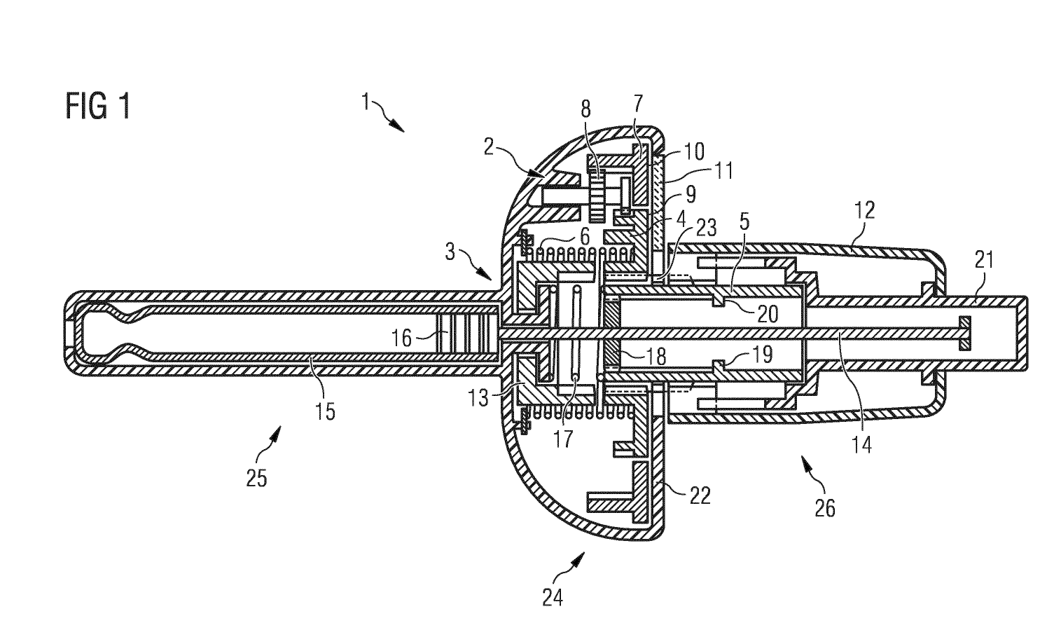

FIG. 1 shows a cross-sectional view of a drug delivery device with a connecting member being in its dose setting position.

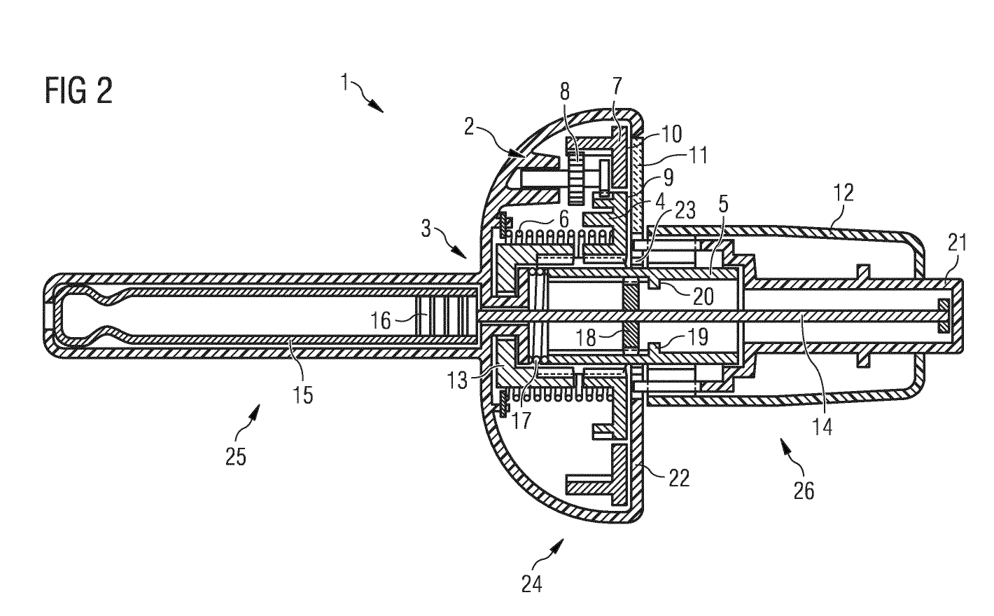

FIG. 2 shows a cross-sectional view of the drug delivery device with the connecting member being in its dose dispensing position.

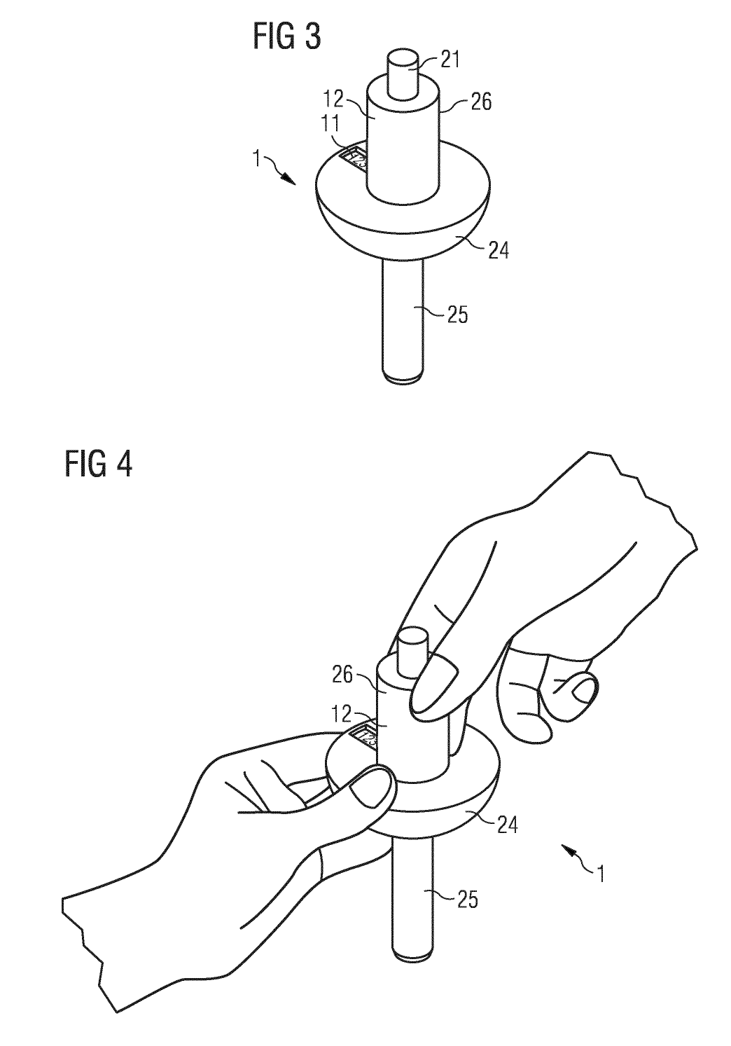

FIG. 3 shows a perspective view of the drug delivery device.

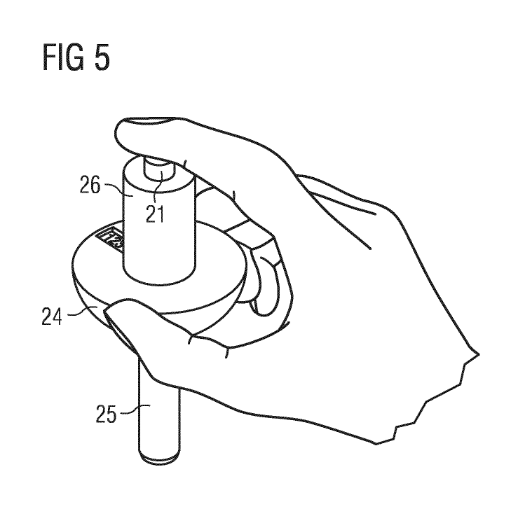

FIG. 4 shows a perspective view of the handling of the drug delivery device during a dose setting operation.

FIG. 5 shows a perspective view of the handling of the drug delivery device during a dose delivery operation.

FIG. 1 shows a cross-sectional view of a drug delivery device 1. The drug delivery device 1 is an injection device. Moreover, the drug delivery device 1 is a variable dose device. The drug delivery device 1 may be a reusable or a disposable device.

The drug delivery device 1 comprises a counting mechanism 2. The drug delivery device 1 further comprises a drive mechanism 3. The counting mechanism 2 is configured to cooperate with the drive mechanism 3. In particular, the counting mechanism 2 comprises some elements that also serve a function in the drive mechanism 3.

The counting mechanism 2 comprises a first counting member 4, a connecting member 5 and a spring member 6. The connecting member 5 has a dose setting position and a dose dispensing position. FIG. 1 shows the connecting member 5 in the dose setting position.

The first counting member 4 comprises a first counting wheel. The first counting member 4 is a units wheel. The counting mechanism 3 further comprises a second counting member 7. The second counting member 7 is a counting wheel, e.g. a tens wheel. Moreover, the counting mechanism 3 comprises a gearing member 8. The gearing member 8 may be an escapement gearing. The gearing member 8 is configured such that a complete rotation of the first counting member 4 is transferred to a rotation of the second counting member 7 by a predetermined angle. Each of the first and the second counting member 4, 7 comprises digits arranged on one of its surfaces 9, 10. The first and the second counting member 4, 7 are arranged such that at least a part of the first counting member 4 and a part of the second counting member 7 is visible in a window 11. Thereby, the counting members 4, 7 are configured to display a number corresponding to the number of currently set doses of the drug delivery device 1.

The spring member 6 comprises a torsion spring which is connected to the first counting member 4. The spring member 6 is preloaded such that it tends to move the first counting member 4 in a first rotational direction. The spring member 6 is arranged such that it is tensed when the first counting member 4 is rotated in the second rotational direction and that it releases energy when the first counting member 4 is rotated in a first rotational direction. The second rotational direction is opposite to the first rotational direction.

The spring member 6 tends to move the first counting member 4 in the first rotational direction wherein, in the dose setting position, the connecting member 5 impedes a rotation of the first counting member 4 in the first rotational direction, thereby preventing the spring member 6 from rotating the first counting member 4 in the first rotational direction. In the dose dispensing position, the connecting member 5 permits the spring member 6 to rotate the first counting member 4 in the first rotational direction.

In the dose setting position, the connecting member 5 is engaged with the first counting member 4 such that the first counting member 4 is rotationally locked to the connecting member 5.

Further, the drug delivery device 1 comprises a control member 12. The control member 12 is a sleeve member. The connecting member 5 is arranged at least partly inside the control member 12. In the dose setting position, the control member 12 is engaged with the connecting member 5 such that the connecting member 5 follows a rotation of the control member 12. As the connecting member 5 is engaged with the first counting member 4 in the dose setting position, the first counting member 4 is rotationally locked to the control member 12 when the connecting member 5 is in the dose setting position.

Moreover, the drive mechanism 3 comprises a drive member 13 and a piston rod 14. The drive member 13 is configured to move the piston rod 14 in a distal direction. Further, the drug delivery device 1 comprises a cartridge 15 comprising a drug and a bung 16. The piston rod 14 is configured to move the bung 16 further in the distal direction into the cartridge 15, thereby expelling a dose of the drug from the cartridge 15.

In the dose setting position of the connecting member 5, the connecting member 5 is disengaged from the drive member 13. Accordingly, a rotation of the connecting member 5 is not transferred into a movement of the drive member 13.

Further, the drive member 13 may be a nut member. The drive member 13 comprises a thread on its inner surface. The piston rod 14 may be a lead screw having a thread on its outer surface. The thread of the piston rod 14 is engaged with the thread of the drive member 13. Accordingly, a rotation of the drive member 13 is transferred into a movement of the piston rod 14 along a longitudinal axis of the drug delivery device 1.

Further, the drug delivery device 1 comprises a return member 17 tending to move the connecting member 5 to the dose setting position. The return member 17 is a spring member. The return member 17 is arranged between the drive member 13 and the connecting member 5.

Further, the counting mechanism 2 comprises a last dose member 18. The last dose member 18 is a last dose nut. The last dose member 18 is engaged with the piston rod 14 and with the connecting member 5. The last dose member 18 is arranged inside the connecting member 5. Further, the connecting member 5 comprises a last dose member stop 19. The last dose member stop 19 comprises a protrusion 20 protruding towards the inside of the connecting member 5. A rotation of the connecting member 5 in the second rotational direction is prevented when the last dose member 18 abuts the last dose member stop 19.

Further, the drug delivery device 1 comprises a trigger member 21 configured to move the connecting member 5 from the dose setting position to the dose dispensing position when the trigger member 21 is operated. The trigger member 21 comprises a button.

The trigger member 21 is movable between a first and a second position. The first position of the trigger member 21 is a more proximal position than the second position of the trigger member 21. The trigger member 21 is in its first position when the connecting member 5 is in its dose setting position. The trigger member 21 is in its second position when the connecting member 5 is in its dose dispensing position.

The trigger member 21 abuts a proximal end of the connecting member 5 in its first and its second position. Alternatively, the trigger member 21 may be arranged to be spaced apart from the proximal end of the connecting member 5 in the first position.

When the trigger member 21 is moved in the distal direction, the connecting member 5 is also moved in the distal direction as the trigger member 21 is configured to push the connecting member 5 into the dose dispensing position. When a user releases the trigger member 21, the return member 17 moves the connecting member 5 back in the proximal direction into the dose setting position. Simultaneously, the return member 17 moves the trigger member 21 from its second position to its first position when the user releases the trigger member 21.

Further, the drug delivery device 1 comprises a body 22. The counting mechanism 2 is arranged at least partly inside the body 22. The trigger member 21 protrudes beyond a proximal end of the body 22. The body 22 comprises the window 11. The first and the second counting member 4, 7 are configured to show a number corresponding to the number of currently set doses in the window 11.

Moreover, the body 22 comprises an engaging member 23. The engaging member 23 is configured to be engaged with the connecting member 5 in the dose setting position. When the engaging member 23 is engaged with the connecting member 5, a rotation of the connecting member 5 in the first rotational direction is prevented. The engaging member 23 may comprise a detent. The engaging member 23 is configured to be engaged with the connecting member 5 such that a rotation of the connecting member 5 in the first rotational direction is impeded while a rotation of the connecting member 5 in the second rotation direction is permitted.

The engaging member 23 may allow rotation of the connecting member 5 in the first direction, for example to reduce the set dose.

The drug delivery device 1 is configured to carry out a priming operation. Priming is the act of preparing the drug delivery device 1 for a first use. Thereby, a small dose of the drug is set and delivered into the air. Thereby, any play and other mechanical tolerances in the drug delivery device 1 are removed and the components are placed into suitable compression and tension. Moreover, the drug delivery device 1 is configured to carry out safety shots. In a safety shot, the user sets one or more small doses of the drug and dispenses the drug into the air before each injection to ensure that a needle through which the drug is expelled is not blocked.

For both, a priming operation of the drug delivery device 1 and for a safety shot operation, the user will set small doses of the drug and inject the dose into the air. The function of the drug delivery device 1 in a priming or a safety shot operation is identical to the function of the drug delivery device 1 in a regular dose setting operation and a regular dose dispensing operation and will become apparent when discussing the dose setting and the dose dispensing operation in more detail.

To set a dose, a user rotates the control member 12. In the dose setting position, the connecting member 5 is rotationally locked to the control member 12. Thus, the connecting member 5 follows a rotation of the control member 12. As the last dose member 18 is threadedly engaged with the connecting member 5, the last dose member 18 is moved axially in the proximal direction relative to the connecting member 5 when the connecting member 5 is rotated. Further, the connecting member 5 is engaged with the first counting member 4 such that the first counting member 4 rotates when the connecting member 5 is rotated.

During the dose setting operation, the control member 12 is rotated in the second rotational direction. Thus, the connecting member 5 and, therefore, the first counting member 4 are also rotated in the second rotational direction. In particular, as the first counting member 4 is rotated in the second rotational direction, the counted number displayed in the window 11 is incremented.

The engaging member 23 of the body 22 is engaged with the connecting member 5 in the dose setting position. Thus, the connecting member 5 is prevented from rotating in the first rotational direction under the force which is exerted by the spring member 6 biasing the first counting member 4.

Moreover, the drug delivery device 1 may comprise a maximum dose limiter (not shown) which limits the amount of the dose that can be set during a dose setting operation. Further, the drug delivery device 1 may comprise a minimum dose limiter (not shown) which defines a minimum amount of the dose that has to be set before the dose is permitted to be dispensed.

When the set dose is increased, the last dose member 18 moves axially away from the distal end of the drug delivery device 1 as the connecting member 5 is rotated relative to the last dose member 18. Thereby, the last dose member 18 is screwed in the proximal direction along the thread of the connecting member 5. If the user attempts to set a greater dose than the volume remaining in the cartridge 15, the last dose member 18 interferes with the last dose member stop 19 on the inside of the connecting member 5. In particular, the last dose member 18 runs axially against the protrusion 20 defined by the last dose member stop 19. Alternatively, the last dose member stop 19 may form a rotary hard stop into which the last dose member 18 is rotated.

As long as the connecting member 5 is in its dose setting position, a user may rotate the control member 12 in the first rotational direction, thereby a resistance due to the engagement of the engaging member 23 with the connecting member 5 has to be overcome. When the control member 12 is rotated in the first rotational direction, the connecting member 5 and, thus, the first counting member 4 follow the rotation in the first rotational direction. Thereby, the set dose is decreased. Thus, the user is enabled to correct an accidental setting of a too large dose before initiating a dose delivery operation.

FIG. 2 shows a cross-sectional view of the drug delivery device 1 wherein the connecting member 5 is in the dose dispensing position.

To inject a dose, a user pushes the trigger member 21 in the distal direction such that the trigger member 21 moves to its second position. Thereby, the trigger member 21 moves the connecting member 5 in the distal direction and the connecting member 5 is moved from its dose setting position to its dose dispensing position. In the dose dispensing position, the connecting member 5 is disengaged from the engaging member 23 of the body 22. Further, in the dose dispensing position, the connecting member 5 is engaged with the drive member 13 such that the drive member 13 is rotationally locked with the connecting member 5.

When the trigger member 21 is arranged at a small distance from the connecting member 5 in its first position, it first travels over the small distance without moving the connecting member 5 before it abuts and then pushes the connecting member 5 to its dose dispensing position. The small distance is in the range of 0.2 mm to 2 mm.

When the trigger member 21 is moved distally into its second position, the return member 17 is compressed. The trigger member 21 is designed to move in the distal direction over a predetermined distance. The predetermined distance is in the range of 2 to 10 mm.

When the trigger member 21 is moved to its second position, it engages with the body 22 and the control member 12, thereby locking the control member 12 against a rotation relative to the body 22.

In the dose dispensing position of the connecting member 5, the spring member 6 is enabled to move the first counting member 4 in the first rotational direction. As the first counting member 4 is rotationally locked with the connecting member 5, the connecting member 5 also rotated in the first rotational direction. Further, the connecting member 5 is rotationally locked with the drive member 13 in the dose dispensing position such that the drive member 13 is also rotated into the first rotational direction. As the drive member 13 is threadedly engaged with the piston rod 14, a rotation of the drive member 13 in the first rotational direction moves the piston rod 14 in the distal direction. Thereby, the set dose of the drug is expelled from the cartridge 15. Accordingly, the energy stored in the spring member 6 during the dose setting operation is used to drive the piston rod 14 in the distal direction during the dose delivery operation. The energy is transferred from the spring member 6 to the piston rod 14 via the first counting member 4, the connecting member 5 and the drive member 13.

As both the piston rod 14 and the connecting member 5 are rotated in the first rotational direction in the dose dispensing operation, the last dose member 18 which is threadedly engaged with the piston rod 14 and with the connecting member 5 follows the rotation in the first rotational direction such that it is not moved relative to the connecting member 5.

If the axial force on the trigger member 21 is removed, the trigger member 21 returns to its first position due to the force exerted by the return member 17. Simultaneously, the return member 17 moves the connecting member 5 to its dose setting position. When the trigger member 21 is moved to its first position, the connecting member 5 is engaged with the engaging member 23 of the body 22, thereby preventing further injection. Moreover, the control member 12 is disengaged from the body 22 such that it can be rotated again by the user to adjust the set dose. Thereby, the user is allowed to interrupt a dose dispensing operation and to split a set dose. During an interruption of the dose dispensing operation, the dose can be amended by rotating the control member 12 which corresponds to carrying out another dose setting operation. When the trigger member 21 is pressed, the dose dispensing operation is restarted.

Moreover, the drug delivery device 1 may comprise a feedback mechanism (not shown). The feedback mechanism indicates the end of a dose dispensing operation by an audible feedback which is provided when the set dose is completely dispensed. The audible feedback is created by two elements (not shown) moving relative to one another near or at the end of the operation. Additionally or alternatively to the audible feedback, the feedback mechanism may provide a tactile feedback or a visible feedback.

The hold time is the period of time from the moment when the drive mechanism 3 has stopped moving to the moment when the dose is fully delivered and the user can remove the needle without affecting the delivered dose volume. In the present mechanism, the speed of the dose delivery operation is defined by the spring member 6. To reduce the hold time, the spring member 6 is configured such that the injection speed is constant during the dose dispensing operation.

FIG. 3 shows a perspective view of the drug delivery device 1. FIG. 4 shows a perspective view of the handling of the drug delivery device 1 in a dose setting operation. FIG. 5 shows a perspective view of the handling of the drug delivery device 1 during a dose delivery operation.

The drug delivery device 1 allows for an ergonomic grip. In particular, the drug delivery device 1 has a reduced length such that users with small hands are also enabled to operate the device 1. As can be seen in FIG. 3, the window 11 is arranged on the face of the body 22 facing in a proximal direction. Thereby, it is easy for a user to view the number displayed in the window 11 while concurrently setting a dose. Moreover, the window 11 is visible to the user during a dose delivery operation. This is in particular important if the user intends to split the dose by interrupting the dose delivery operation.

The drug delivery device 1 comprises a gripping portion 24. The gripping portion 24 is the portion of a maximum diameter. The gripping portion 24 is arranged in a middle part of the drug delivery device 1. The drug delivery device 1 further comprises a distal end portion 25 and a proximal end portion 26. The gripping portion 24 is arranged between the distal end portion 25 and the proximal end portion 26.

The gripping portion 24 serves two functions. The gripping portion 24 provides an easy grip even for users having small hands or impaired dexterity. Moreover, the gripping portion 24 provides space for parts of the counting mechanism 2 and of the drive mechanism 3 such that the widened gripping portion 24 allows reducing the total length of the drug delivery device 1.

In particular, the drive member 13, the first counting member 4, the second counting member 7, the gearing member 8, the spring member 6 and the return member 17 are arranged completely inside the gripping portion 24 of the body 22.

### REFERENCE NUMERALS

1 drug delivery device
2 counting mechanism
3 drive mechanism
4 first counting member
5 connecting member
6 spring member
7 second counting member
8 gearing member
9 surface of the first counting member
10 surface of the second counting member
11 window
12 control member
13 drive member
14 piston rod
15 cartridge
16 bung
17 return member
18 last dose member
19 last dose member stop
20 protrusion
21 trigger member
22 body
23 engaging member
24 gripping portion
25 distal end portion
26 proximal end portion

1. Counting mechanism (2) for a drug delivery device (1) comprising
a first counting member (4) arranged for displaying a counted number and being biased to move in a first rotational direction, and
a connecting member (5) having a dose setting position and a dose dispensing position,
wherein, in the dose setting position, the connecting member (5) impedes a rotation of the first counting member (4) in the first rotational direction, and
wherein, in the dose dispensing position, the connecting member (5) permits the first counting member (4) to rotate in the first rotational direction.

2. Counting mechanism (2) according to claim 1,
wherein, in the dose setting position, the connecting member (5) permits a rotation of the first counting member (4) in a second rotational direction.

3. Counting mechanism (2) according to one of the preceding claims,
wherein the connecting member (5) is rotationally locked with the first counting member (4) in the dose setting position and in the dose dispensing position.

4. Counting mechanism (2) according to one of the preceding claims,
wherein the connecting member (5) is movable along a longitudinal axis of the mechanism between the dose setting position and the dose dispensing position.

5. Counting mechanism (2) according to one of the preceding claims,
comprising a trigger member (21) configured to move the connecting member (5) from the dose setting position to the dose dispensing position when the trigger member (21) is operated.

6. Counting mechanism (2) according to one of the preceding claims,
comprising a return member (17) tending to move the connecting member (5) to the dose setting position.

7. Counting mechanism (2) according to one of the preceding claims,
comprising a spring member (6) which biases the first counting member (4) to move in the first rotational direction.

8. Counting mechanism (2) according to one of the preceding claims,
comprising an energy storing member (6) configured to store energy when the first counting member (4) is rotated in the second rotational direction.

9. Counting mechanism (2) according to claim 8,
wherein the energy storing member (6) is configured to release the stored energy and thereby to move the first counting member (4) in the first rotational direction when the connecting member (5) is in the dose dispensing position.

10. Counting mechanism (2) according to one of the preceding claims,
wherein the counting mechanism (2) further comprises a second counting member (7), and
wherein the second counting member (7) is coupled to the first counting member (4) via a gearing member (8).

11. Drug delivery device (1) comprising a counting mechanism according to one of the preceding claims and a body.

12. Drug delivery device (1) according to claim 11,
comprising a piston rod (14) and a drive member (13) configured to move the piston rod (14),
wherein the connecting member (5) is engaged with the drive member (13) in the dose dispensing position such that the drive member (13) is rotationally locked with the connecting member (5) in the dose dispensing position.

13. Counting mechanism according to claim 12,
wherein the connecting member (5) is disengaged from the drive member (13) in the dose setting position such that the connecting member (5) is permitted to rotate relative to the drive member (13) in the dose setting position.

14. Drug delivery device (1) according to one of claims 11 to 13,
wherein the connecting member (5) is engaged with the body (22) in the dose setting position, and
wherein the connecting member (5) is disengaged from the body (22) in the dose dispensing position.

15. Drug delivery device (1) according to one of claims 11 to 14,
comprising a distal end portion (25), a gripping portion (24) and a proximal end portion (26)
wherein the gripping portion (24) is a portion of a maximum diameter in the drug delivery device (1), and
wherein the gripping portion (24) is arranged between the distal end portion (25) and the proximal end portion (26).
